(19) United States
(12) Patent Application Publication
(10) Pub. No.: US 2021/0267785 A1
(43) Pub. Date: Sep. 2, 2021

(54) BREATHING ACCESSORY

(71) Applicant: Warren R Ebener, JR., Cameron, SC (US)

(72) Inventor: Warren R Ebener, JR., Cameron, SC (US)

(21) Appl. No.: 16/804,059

(22) Filed: Feb. 28, 2020

(51) Int. Cl. A61F 5/08

(52) U.S. Cl. CPC A61F 5/08

(57) ABSTRACT

A manually formable member applies a lateral force to each of the nostrils to reduce or prevent the lateral (side) walls of the nose from collapsing inwardly when the wearer inhales. A first end of the breathing accessory is placed in one nostril, and the opposite end is placed within a second or opposite nostril. An arcuate portion of the breathing accessory is positioned over the bridge of the wearer's nose to hold the device in place. The breathing accessory is so constructed of a formable member that can be manually manipulated to a desired shape to provide a custom fit for the wearer.

### BACKGROUND OF THE INVENTION

Nasal breathing delivers approximately seventy percent of airflow to the lungs according to one source. Nasal airway obstruction can limit airflow. An appreciable percentage of people suffer from nasal airway obstruction, which can lead to other health problems and quality of life issues.

A slight narrowing of the nasal airway can lead to significant reduction in airflow. Common blockages may result from problems with the septum, or ridges of bone and tissue inside the nose. One particular problem is a weak lateral wall of the nose. Weak or excessively flexible cartilage in this outer nasal wall may cause it to collapse when a person inhales.

There is a need for a device that is inexpensive and easy to use that improves nasal airflow.

### SUMMARY OF THE INVENTION

The present invention is a manually formable member that applies a lateral force to each of the nostrils to reduce or prevent the lateral (side) walls of the nose from collapsing inwardly when the wearer inhales. A first end of the breathing accessory is placed in one nostril, and the opposite end is placed within a second or opposite nostril. An arcuate portion of the breathing accessory is positioned over the bridge of the wearer's nose to hold the device in place. The breathing accessory is so constructed of a formable member that can be manually manipulated to a desired shape to provide a custom fit for the wearer.

### BRIEF DRAWING DESCRIPTION

FIG. 1 shows the breathing accessory according to an embodiment of the invention in use by a wearer.

FIG. 2 shows a perspective view of a preferred embodiment of the breathing accessory.

FIG. 3 shows an elevation of an embodiment of the breathing accessory demonstrating manual reformation of the breathing accessory to custom fit the breathing accessory for persons having different nasal shapes and sizes.

FIG. 4 is an elevation showing a coated breathing accessory with a manually formable member shown as a phantom and having a coating thereon.

FIG. 5 is a perspective view of an embodiment of the breathing accessory with a pad covering an arcuate midsection of the breathing accessory.

### DESCRIPTION OF PREFERRED EMBODIMENTS

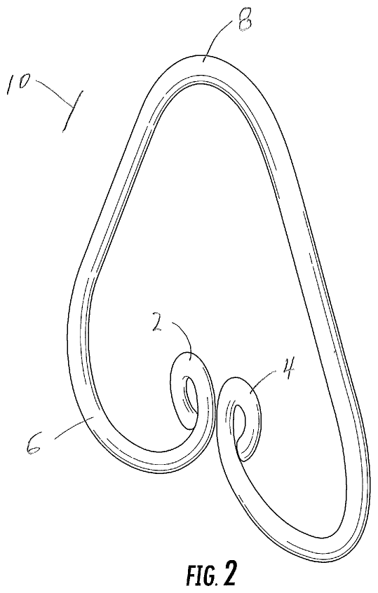

FIG. 2 shows an embodiment of the breathing accessory 10. The breathing accessory has a first end 2 and a second end 4. Between the first end and the second end is an arcuate midsection 8.

The first end 2 and the second end 4 are formed in an arcuate shape. The arcuate shapes of the first end and the second end are formed so that the first end and second end curve toward a remaining portion of the manually formable member 6. The first end and second end are arcuate and curved so that they substantially touch a portion of the manually formable member that is near the first end and second end. By “substantially touching” is meant that the terminus of the first end and terminus of the second end touch, or come very close to touching, a portion of the manually formable member that is near the first end and the second end, but the gap between the terminus of the first end and the terminus of the second end are not sufficiently large so as to receive tissue therein that would trap or pinch the nasal tissue.

Moving away from the terminus of the first end 2 and terminus of the second end 4, the manually formable member, in use, may have an arcuate section 12 on each side of the manually formable member 6. Still referring to FIG. 2, the manually formable member on each side extends upwardly to join at the arcuate midsection 6 that is so constructed and arranged to receive a bridge of a wearer's nose 14 within the arcuate midsection of the manually formable member.

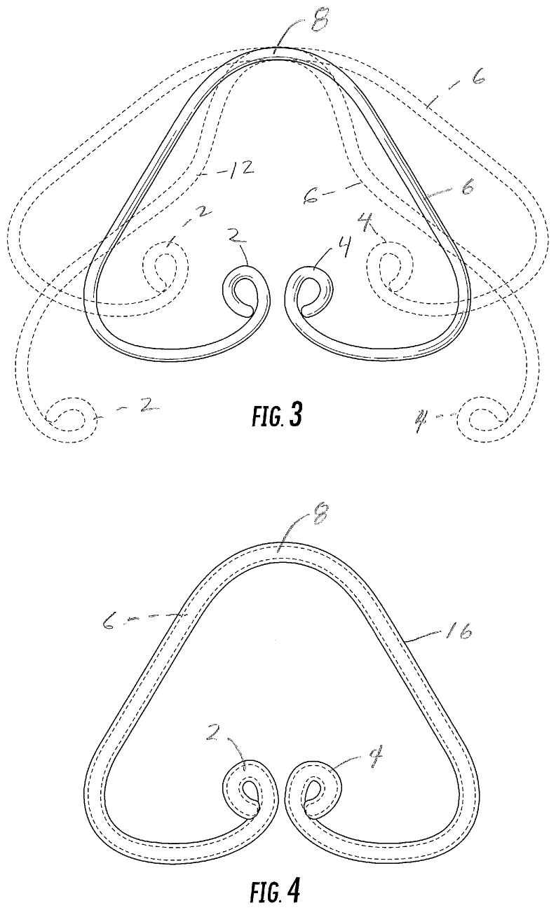

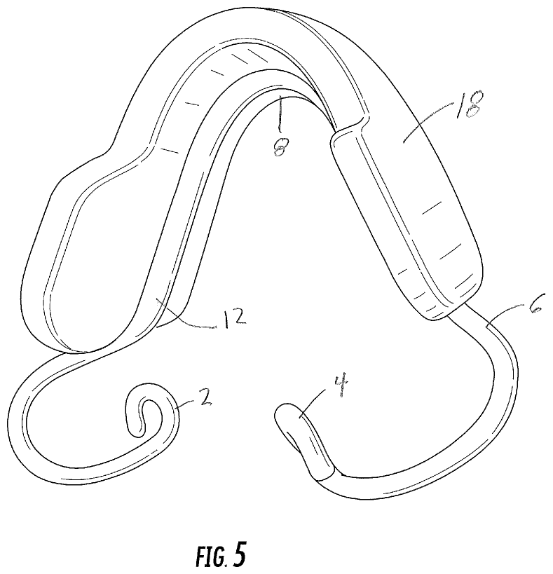

The manually formable member 6 is preferred to be formed in a unitary structure. The manually formable member is preferred to be made in one elongated piece and shaped as described and shown. By “manually formable” it is meant that the manually formable member may be manually (by hand) formed by the wearer to fit the wearer's particular nasal anatomy. As demonstrated by FIG. 3, the manually formable member may be manually pulled laterally to expand the device, or the sides of the manually formable member may be pushed closer to each other to reduce the overall width of the device. The arcuate midsection 6 may be reformed to fit the bridge of the wearer's nose 14 to hold the device in place. A portion of the device between the first end 2 and the arcuate midsection and the portion of the device between the arcuate midsection and the second end 4 may be pushed inwardly with manual force to form an arcuate portion 12 of the manually formable member which aids in holding the device in place over the bridge of the wearer's nose.

In a preferred embodiment, the manually formable member 6 may be constructed from a wire that, once formed, will retain its shape until the wire is manually reformed. The wire may be stainless steel or nitinol that is manually formable.

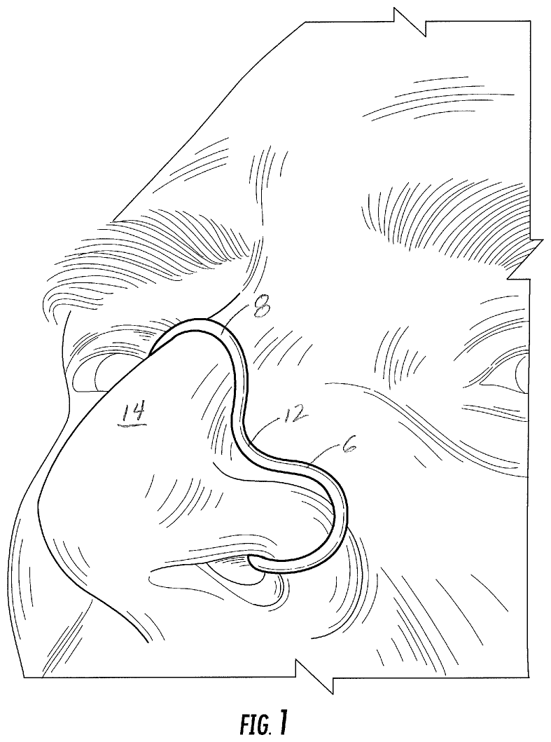

FIG. 1 shows the breathing accessory in position on a wearer's nose 14. The manually formable member 6 of the breathing accessory is shaped by hand to provide a lateral force on the nostrils to hold the nostrils open for efficient breathing. At the same time, the manually formable member has been shaped so that the architecture of the nose 14, and particularly the bridge of the nose, to hold the manually formable member in position.

In FIG. 1, the manually formable member 6 is spread laterally from the position shown in FIG. 2 so that resistance a force is applied to a first nostril and a lateral force is applied to a second nostril. In one application, the ends 2,4 are spread laterally a sufficient distance so that a lateral force is applied to the lateral walls of the nose. The force on the nostrils is opposite, so that the lateral walls of the nostrils are held open, thereby retarding or preventing the nasal walls from collapsing inwardly as the wearer person inhales.

Also as shown in FIG. 1, the arcuate midsection 8 is manually formed to fit over the bridge of the wearer's nose 14. In most cases, the opposing sides 12 of the manually formable member 6 midsection are pushed together for contact with the bridge of the wearer's nose to hold the breathing accessory in place. The sides of the manually formable member should not be pushed toward each other to a degree that will narrow the device to cause discomfort or inhibit airflow. The manually formable member allows the breathing accessory to be manually spread apart at the arcuate midsection as necessary for a proper fit.

For most wearers, the sides 12 of the manually formable member 6 that are between the first end and the arcuate midsection on one side and the arcuate midsection on the second end on the opposite side will be formed in an arcuate shape, to provide contact with the lateral or side walls of the nose 14 for further assistance in holding the device in place. The concave sides 12 should not fit so tightly on the wearer's nose so as to cause discomfort to the wearer or inhibit nasal airflow.

The arcuate ends 2,4 of the manually formable member 6 are inserted into the nostrils of the wearer's nose 14. The shape of the arcuate ends presents no sharp surfaces that may be harmful to the wearer's anatomy. The arcuate ends of the manually formable member are small enough to be positioned in the nostrils without materially inhibiting airflow into the nose, but are large enough to provide sufficient contact with the interior of the nostrils to prevent or retard the lateral walls of the nose from collapsing. The arcuate ends provide resistance to the interior of the nose to prevent or retard the lateral walls of the nose from collapsing, or if the manually formable member is spread sufficiently, a force is applied to the lateral walls of the nose by the arcuate ends.

In a preferred embodiment, the manually formable member 6 is coated with a coating 16. The manually formable member may be a wire that is coated with a soft plastic. The soft plastic may be more comfortable to wear than an exposed metal wire, and has less thermal conductivity than a metal wire. Further, coating the wire with a plastic or other coating reduces or prevents corrosion of the wire.

As shown in FIG. 5, a pad 18 may be placed over the arcuate midsection 8 or other portions of the breathing accessory, including the sides 12 of the breathing accessory. The pad may be formed of common materials from which pads are formed, such as a foam material or a textile material. Nose pads may be made of soft silicon. Nose pads may comprise an adhesive for connection to the breathing accessory, or a breathing accessory may be inserted through a sleeve formed in the nose pad. The manually formable member 6 holds the pad in the desired shape. The pad is sufficiently flexible to allow the manually formable member to be shape formed as described herein.

What is claimed is:

1. A breathing accessory, comprising
a manually formable member, the manually formable member comprising a first end and a second end, wherein a portion of the manually formable member that is proximate to the first end and a portion of the manually formable member that is proximate to the second end are formed in an arcuate shape with the first end of the manually formable member substantially touching the portion of the manually formable member that is near the first end and the second end of the manually formable member substantially touching the portion of the manually formable member that is near the second end, the manually formable member comprising an arcuate mid-section that is so constructed and arranged to receive a bridge of a wearer's nose within the arcuate mid-section of the manually formable member.

2. A breathing accessory as described in claim 1, wherein the manually formable member comprises a first concave section that is positioned between the first end and the arcuate mid-section of the manually formable member and a second concave section that is positioned between the second end and the arcuate mid-section of the manually formable member.

3. A breathing accessory as described in claim 1, wherein, in use, the manually formable member is so constructed and arranged to apply resistance to a lateral wall of a first nostril and resistance to a lateral wall of a second nostril.

4. A breathing accessory as described in claim 1, wherein, in use, the first end of the manually formable member is so constructed and arranged for positioning in a first nostril and applies resistance to a lateral wall of the first nostril and the second end of the manually formable member is so constructed and arranged for positioning in a second nostril and applies resistance to a lateral wall of the second nostril.

5. A breathing accessory as described in claim 1, wherein the manually formable member comprises a length of wire.

6. A breathing accessory as described in claim 1, wherein the manually formable member comprises a length of wire coated with plastic.

7. A breathing accessory as described in claim 1, wherein the portion of the manually formable member that is proximate the first end is so constructed and arranged to fit within a first nostril of a wearer and the portion of the manually formable member that is proximate the second end is so constructed and arranged to fit within a second nostril of a wearer.

8. A breathing accessory as described in claim 1, wherein the arcuate mid-section that is so constructed and arranged to receive a bridge of a wearer's nose comprises a pad.

9. A breathing accessory as described in claim 1, wherein, in use, the first end of the manually formable member is so constructed and arranged for positioning in a first nostril and applies a force and resistance to a lateral wall of the first nostril and the second end of the manually formable member is so constructed and arranged for positioning in a second nostril and applies force and resistance to a lateral wall of the second nostril.
